(19) United States
(12) Patent Application Publication
(10) Pub. No.: US 2026/0248491 A1
(43) Pub. Date: Aug. 27, 2026

(54) SYSTEMS AND METHODS FOR IDENTIFICATION, MODE-SETTING, AND/OR TRACKING OF SURGICAL INSTRUMENTS

(71) Applicant: Medtronic Xomed LLC, Minneapolis, MN (US)

(72) Inventors: Manfred K. Luedi, Zwingen (CH); Christopher L. Fair, Jacksonville Beach, FL (US); Stacey S. Dexter, Keller, TX (US); Vikram A. Garadi, Keller, TX (US)

(21) Appl. No.: 18/714,476

(22) PCT Filed: Jan. 10, 2023
(86) PCT No.: PCT/US2023/010479

§ 371 Date: May 29, 2024

Related U.S. Application Data
(60) Provisional application No. 63/298,446, filed on Jan. 11, 2022.

(51) Int. Cl. A61B 17/00; A61B 34/20; G16H 40/63

(52) U.S. Cl. CPC A61B 17/00; A61B 34/20; G16H 40/63; A61B 2017/00199; A61B 2017/00225; A61B 2017/0046; A61B 2017/00973

(57) ABSTRACT

A surgical system includes a surgical handpiece having a handle housing, a connector extending from the handle housing and adapted to connect the surgical handpiece to a console, a tool releasably coupled with the handle housing, and orientation sensor disposed within the handle housing. The orientation sensor is configured to facilitate determination of an orientation of the surgical handpiece from between at least a use orientation and a change orientation. The surgical system may further include a console wherein a processor of the console determines the orientation.

### CROSS-REFERENCE TO RELATED APPLICATIONS

This application claims the benefit of and priority to U.S. Provisional Patent Application No. 63/298,446, filed on Jan. 11, 2022, the entire contents of which are hereby incorporated herein by reference.

### FIELD

The present disclosure relates to surgical systems and methods and, more specifically, to systems and methods for identification, mode-setting, and/or tracking of one or more surgical instruments in a surgical system.

### BACKGROUND

Surgical handpieces are used in many different surgical procedures to enable the manipulation, control, and/or actuation of a surgical tool attached to the surgical handpiece. Powered surgical handpieces, for example, may be used to drive surgical drills, blades, and/or other cutting tools to facilitate performing various different surgical cutting functions including drilling, tapping, resection, dissection, debridement, shaving, sawing, pulverizing, and/or shaping of anatomical tissue including bone. Powered surgical handpieces may be driven by an electric motor, a pneumatic motor, an ultrasonic transducer, or may be driven in any other suitable manner. One or more of such powered surgical handpieces may connect to a console that supplies power and/or control signals thereto.

During a surgical procedure, one or more powered surgical handpieces (of similar or different type) may be utilized each with an appropriate surgical tool mounted thereto to facilitate performing one or more surgical tasks. In many procedures, the surgical tool(s) are removed and different surgical tool(s) are attached to the powered surgical handpiece(s) to facilitate performing different surgical task(s). Thus, during a surgical procedure, multiple powered surgical handpieces and/or multiple surgical tools for each powered surgical handpiece may be utilized with a single console or multiple consoles.

### SUMMARY

As used herein, the term “distal” refers to the portion that is being described which is farther from an operator, while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. To the extent consistent, any of the aspects described herein may be used in conjunction with any or all of the other aspects described herein.

Provided in accordance with aspects of the present disclosure is a surgical system including a surgical handpiece having a handle housing, a connector (e.g., a cable and/or hose) extending from the handle housing and adapted to connect the surgical handpiece to a console, a tool releasably coupled with the handle housing, and an orientation sensor disposed within the handle housing. The orientation sensor is configured to facilitate determination of an orientation of the surgical handpiece from between at least a use orientation and a change orientation.

In an aspect of the present disclosure, the orientation sensor includes at least one of an accelerometer, a gyroscope, or a magnetometer.

In another aspect of the present disclosure, the orientation sensor is further configured to facilitate determination of the orientation of the surgical handpiece from between at least the use orientation, the change orientation, and a standby orientation.

In still another aspect of the present disclosure, the surgical handpiece further includes an output device configured to provide at least one of an audible output, a visual output, or a haptic output.

In yet another aspect of the present disclosure, the surgical system further includes the console. The orientation sensor, in such aspects, is configured to transmit orientation sensor data to the console and a processor of the console is configured to determine the orientation of the surgical handpiece based at least in part on the orientation sensor data.

In still yet another aspect of the present disclosure, the processor of the console is further configured to set or maintain the surgical handpiece to an active mode when the determined orientation is the use orientation and/or set or maintain the surgical handpiece to a safe mode when the determined orientation is the change orientation. In aspects, at least some functionality of the surgical handpiece is inhibited in the safe mode.

Another surgical system provided in accordance with aspects of the present disclosure includes a console and a plurality of surgical handpieces connected to the console. Each surgical handpiece includes a handle housing, a connector (e.g., a cable and/or hose) extending from the handle housing and connecting the surgical handpiece to the console, a tool releasably coupled with the handle housing, and an orientation sensor disposed within the handle housing. The console includes a processor configured to receive orientation sensor data from the orientation sensor of each surgical handpiece of the plurality of surgical handpieces and to determine at least one active surgical handpiece of the plurality of surgical handpieces and at least one inactive surgical handpiece of the plurality of surgical handpieces based on the orientation sensor data.

In an aspect of the present disclosure, the console enables remote control of the at least one active surgical handpiece and inhibits remote control of the at least one inactive surgical handpiece. The remote control may include control at the console and/or control at an accessory attached to the console, e.g., a footswitch.

In another aspect of the present disclosure, the processor of the console is configured to determine whether each surgical handpiece of the plurality of surgical handpieces is in motion or has recently been in motion based on the orientation sensor data to determine the at least one active surgical handpiece and the at least one inactive surgical handpiece.

In still another aspect of the present disclosure, each surgical handpiece of the plurality of surgical handpieces further includes an output device configured to provide at least one of an audible output, a visual output, or a haptic output. In such aspects, the processor of the console may be configured to direct the output device of the at least one active surgical handpiece to output an indication that the at least one active surgical handpiece is active.

In yet another aspect of the present disclosure, the processor of the console is configured set or maintain the at least one inactive surgical handpiece to a safe mode.

Another surgical system provided in accordance with aspects of the present disclosure includes a console and a surgical handpiece connected to the console. The surgical handpiece includes a handle housing, a connector (e.g., a cable and/or hose) extending from the handle housing and connecting the surgical handpiece to the console, a tool releasably coupled with the handle housing, and an orientation sensor disposed within the handle housing. The console includes a processor configured to receive orientation sensor data from the orientation sensor of the surgical handpiece and to determine a trajectory of motion of the surgical handpiece based thereon.

In an aspect of the present disclosure, the orientation sensor includes at least one of an accelerometer, a gyroscope, or a magnetometer.

In another aspect of the present disclosure, the processor of the console is further configured to determine, based on further orientation sensor data received from the orientation sensor of the surgical handpiece, whether the surgical handpiece deviates from the determined trajectory.

In still another aspect of the present disclosure, the surgical handpiece includes an output device and the processor of the console is configured to instruct the output device to provide an output indicating that the surgical handpiece has deviated from the determined trajectory when it is determined that the surgical handpiece has deviated from the determined trajectory. The output device may be configured to provide at least one of an audible output, a visual output, or a haptic output.

### BRIEF DESCRIPTION OF THE DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of a surgical system provided in accordance with the present disclosure including a console, a footswitch, and a plurality of powered surgical handpieces;

FIGS. 2A and 2B are side views of various different couplers and tools, respectively, configured for use with the powered surgical handpieces of the surgical system of FIG. 1;

FIG. 3A illustrates a powered surgical handpiece of the surgical system of FIG. 1 including a coupler attached thereto and a tool attached to the coupler, wherein the powered surgical handpiece is disposed in a use orientation;

FIG. 3B illustrates the powered surgical handpiece including the coupler and tool as shown in FIG. 3A, wherein the powered surgical handpiece is disposed in a change orientation;

FIG. 3C illustrates a plurality of powered surgical handpieces of the surgical system of FIG. 1 disposed on a surgical tray in standby orientations;

FIG. 4 is a flow diagram illustrating a process provided in accordance with the present disclosure;

FIG. 5 is a block diagram representing connections between the console, the footswitch, and a plurality of powered surgical handpieces of the system of FIG. 1;

FIG. 6 is a flow diagram illustrating another process provided in accordance with the present disclosure;

FIG. 7 is a perspective view illustrating a powered surgical handpiece of the system of FIG. 1 including a coupler attached thereto and a tool attached to the coupler, shown in use performing a surgical task on tissue of a patient; and

FIG. 8 is a flow diagram illustrating still another process provided in accordance with the present disclosure.

### DETAILED DESCRIPTION

The present disclosure relates to systems and methods for identification, mode-setting, and/or tracking of one or more surgical instruments in a surgical system. Although the aspects and features of the present disclosure are described below with reference to a surgical system including a console and one or more powered surgical handpieces each including a coupler releasably engaged therewith and a tool releasably engaged with the coupler, it is understood that the aspects and features of the present disclosure are equally applicable for use with other suitable surgical systems such as, for example, other powered surgical systems, mechanical surgical systems, energy-based surgical systems, surgical visualization systems, robotic surgical systems, etc.

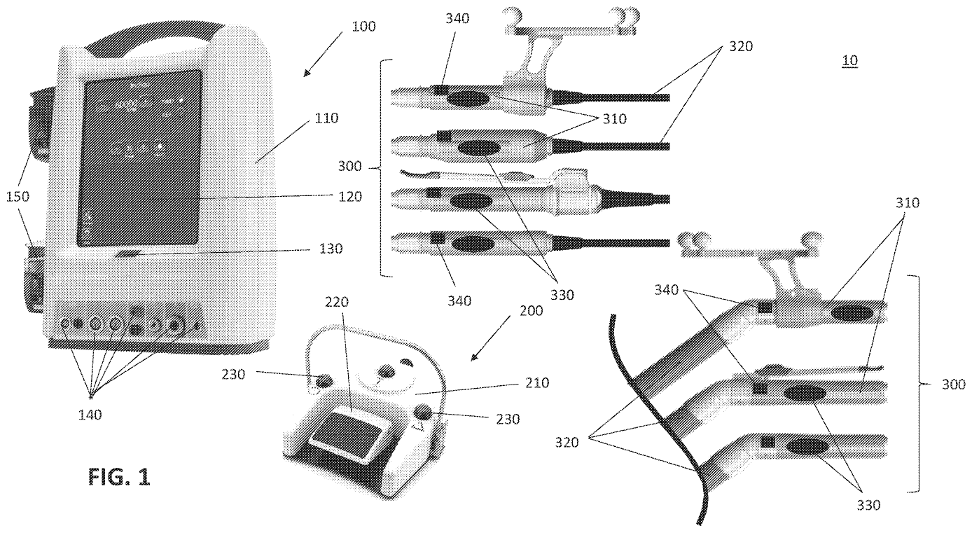

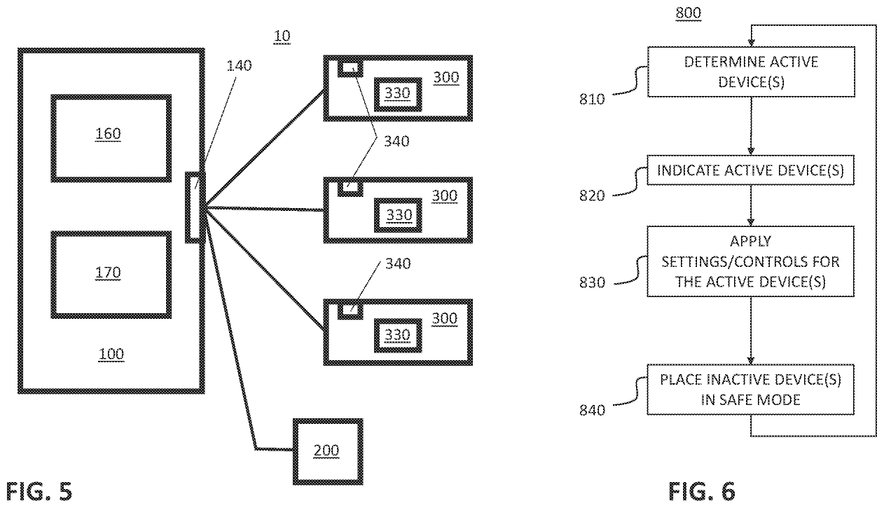

Turning to FIG. 1, a surgical system 10 provided in accordance with the present disclosure includes a console 100, a footswitch 200, and one or more surgical handpieces 300. Console 100 may include an outer housing 110 enclosing the internal operable components of console 100, a touch screen graphical user interface (GUI) 120 to receive user input and display information to the user, an ON/OFF button 130, a plurality of device ports 140, one or more fluid pumps 150, and/or other suitable features. One or more processors 160 (FIG. 5) and associated memory(s) 170 (FIG. 5) are disposed within outer housing 110 and serve to control power and control signals supplied to connected devices through device ports 140, GUI 120; to process user inputs, feedback data, and other information input to console 100; and to control the one or more fluid pumps 150. Suitable hardware and drive mechanisms may be disposed within outer housing 110 to perform the various functions of console 100 and may include, for example, power-generating circuitry, sensor circuitry, motors, pump drivers, etc.

Footswitch 200 is configured to connect to one of the device ports 140 of console 100 and includes a base 210, a foot pedal 220 (including an underlying switch or switches (not explicitly shown), and one or more control buttons 230. A cord (not shown) having a plug at the free end is configured to connect footswitch 200 to one of the device ports 140 of console 100 such that user inputs provided to footswitch 200 are received at console 100, e.g., for controlling settings, actuating, and/or otherwise operating one or more devices associated with console 100.

Surgical handpieces 300 may define various different configurations for use in performing various different surgical tasks, for use in various different procedures, etc. Surgical handpieces 300 may additionally or alternatively include various different internal mechanisms for different types of actuation, e.g., electric motors or suitable internal mechanisms for electric motor-driven actuation, pneumatic motors or suitable internal mechanisms for pneumatic motor-driven actuation, ultrasonic transducers or suitable internal mechanisms for ultrasonically-driven actuation, etc. Further, some surgical handpieces 300 may include different features such as, for example, hand control(s), navigation, rotation, articulation, etc.

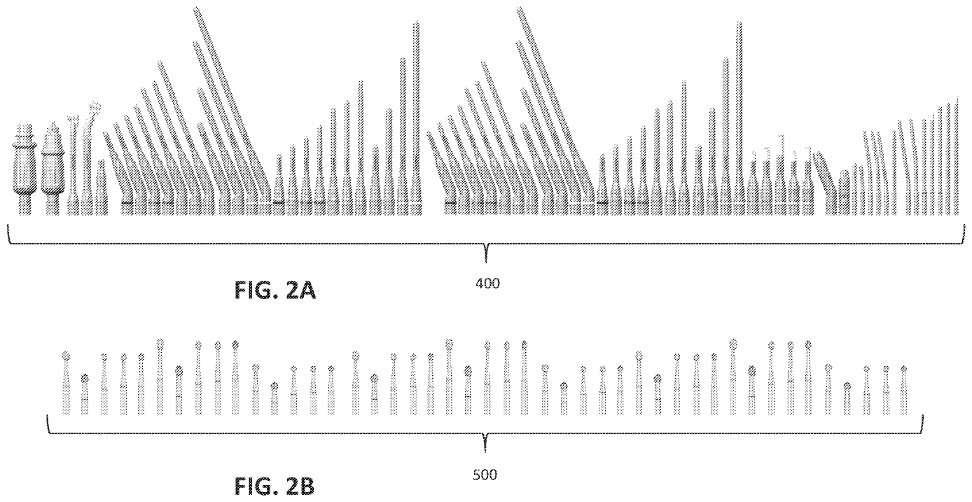

Each surgical handpiece 300 generally includes: a handle housing 310; a connector 320 (e.g., a cable (for electrical connections such as for control, feedback, electric motors, or other energy-based device(s)) and/or hose (for pneumatic devices)) configured to operably connect the handle housing 310 to one of the device ports 140 of console 100 such that power (electrical, pneumatic, and/or other) and/or control signals can be provided to handle housing 310 and/or such that feedback signals can be provided to console 100; an orientation sensor 330 disposed within handle housing 310; and, in aspects, an output device 340 disposed on or within handle housing 310. Some of the handle housings 310 may further include motors, ultrasonic transducers, and/or other mechanisms configured to drive the components attached thereto, e.g., in response to suitable power and/or control signals from console 100. In some other handle housings 310, these drive mechanisms are omitted and such handle housings 310 are driven by suitable drive mechanisms disposed within console 100. In either configuration, each surgical handpiece 300 may be powered to drive an attached tool 500 (FIG. 2B) at selected and/or selectable speeds of from about 100 RPM to about 100,000 RPM, for example.

The orientation sensor 330 of each surgical handpiece 300 may include, for example, one or more accelerometers, one or more gyroscopes, one or more magnetometers, combinations thereof, etc. Each orientation sensor 330 is connected to console 100, e.g., wirelessly or via suitable wiring extending through the corresponding connector 320, to relay sensed orientation data thereto. One of the processors 160 (FIG. 5) of console 100 is configured to determine the orientation of the surgical handpiece 300 based on the sensed orientation data provided thereto, which may be an absolute orientation (e.g., based on a global reference system) or a relative orientation (e.g., based on one or more known orientations, one or more previous orientations, a local reference system, etc.). In aspects, console 100 determines whether the determined orientation of surgical handpiece 300 corresponds (or is sufficiently close, e.g., within 10 degrees or within 20 degrees) to one or more pre-defined orientations of the surgical handpiece 300. The pre-defined orientations may be set at manufacturing, may be user-settable, e.g., during a calibration mode upon connection of the surgical handpiece 300 to console 100, or may be determined in any other suitable manner. The pre-define orientations may include, for example, a use orientation, a change orientation, or a standby orientation, as detailed below.

The orientation sensor 330 of each surgical handpiece 300 is additionally or alternatively configured to detect motion, e.g., by detecting changes in direction, acceleration, or other movement of the handle housing 310. More specifically, the sensed orientation data may be utilized by the processor 160 (FIG. 5) of console 100 to determine whether the surgical handpiece 300 is in motion (and if so, one or more of: a type of motion (linear, rotational, etc.), a direction of motion, a trajectory of motion, a rate of motion (velocity or acceleration), etc.) or whether surgical handpiece 300 is stationary.

The output device 340 of each surgical handpiece 300 may include one or more of a speaker, an LED, a haptic device, etc. and, as noted above, is disposed on or within the corresponding handle housing 310. The output device 340 is connected to console 100, e.g., wirelessly or via suitable wiring extending through the corresponding connector 320, and is configured to receive output signals therefrom to provide an indication to the user via output device 340, e.g., in the form of an audible tone or other audible output, an illuminated LED or other visual output, and/or a vibration or other haptic output. In this manner, output device 340 provides feedback to the user without requiring the user to view or hear output provided at console 100, which may be more remotely located and/or out of view. In other aspects, output device 340 is omitted from surgical handpiece 300 and the output is provided at console 100 or other suitable device.

With additional reference to FIGS. 2A and 2B, each handle housing 310 is configured to releasably receive one or more different couplers 400 and each coupler 400, in turn, is configured to releasably receive and engage one or more different tools 500 to thereby operably couple the engaged tool 500 with the surgical handpiece 300. In aspects, couplers 400 may be omitted and one or more different tools 500 may be received within some or all handle housings 310 in operable engagement with the surgical handpieces 300. More specifically, with a tool 500 operably coupled to a surgical handpiece 300 (either directly or via a coupler 400), the tool 500 is operably coupled with the drive components of the surgical handpiece 300 and/or console 100, e.g., the motors, ultrasonic transducers, and/or other mechanisms, to enable rotational and/or reciprocational driving of the tool 500 for treating, e.g., cutting, tissue.

The various different surgical handpieces 300, couplers 400, and tools 500 enable instrument customization according to surgeon preference, the tissue to be operated on, the anatomical location of the operation, the procedure to be performed, and/or the surgical task(s) to be performed such as, for example, drilling, tapping, resection, dissection, debridement, shaving, sawing, pulverizing, and/or shaping. Additional or alternative tools 500 (with or without couplers 400) are also contemplated such as, for example, tools 500 that provide energy to tissue for treating tissue (e.g., monopolar Radio Frequency (RF) energy, bipolar RF energy, light energy, thermal energy, ultrasonic energy, microwave energy, etc.); tools 500 used for visualization; tools 500 used for illumination; tools 500 used for tracking, tagging, and/or navigation; and/or any other suitable tools 500.

Regardless of the particular surgical handpiece(s) 300, coupler(s) 400, and/or tool(s) 500 utilized, the releasable engagement of couplers 400 and tools 500 with one another and handle housings 310 readily enables switching between various different surgical handpieces 300, couplers 400, and/or tools 500 during a surgical procedure to enable performance of various different surgical tasks.

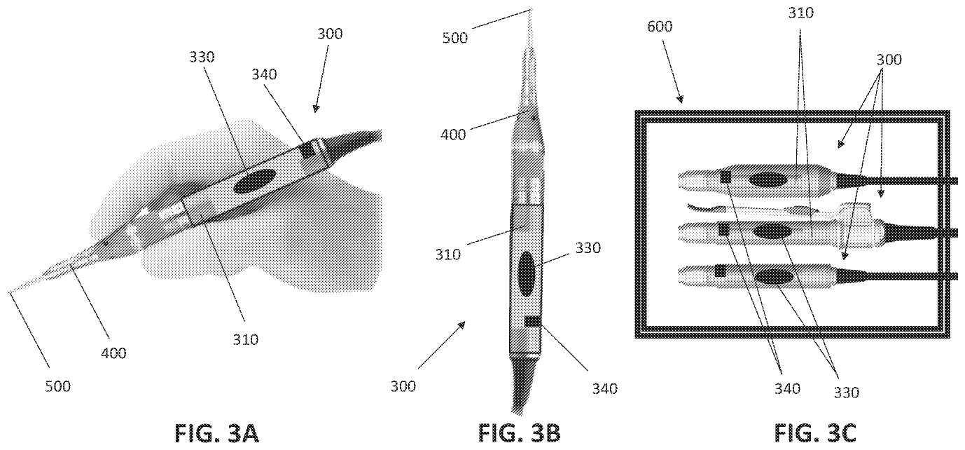

Referring to FIGS. 3A-3C, each surgical handpiece 300 may assume various different and distinguishable orientations depending upon whether the surgical handpiece 300 is actively being manipulated or held, the purpose or intention of the manipulation or holding, whether the surgical handpiece 300 is not being manipulated or held, and/or other factors. For example: as shown in FIG. 3A, during active use of a surgical handpiece 300 to perform a surgical task, e.g., to cut tissue, the surgical handpiece 300 may be oriented in a use orientation wherein the tool 500 at the distal portion of the surgical handpiece 300 is angled downwardly relative to the handle housing 310 at the proximal portion of the surgical handpiece 300; as shown in FIG. 3B, when it is desired to change the tool 500 and/or coupler 400, the surgical handpiece 300 may be disposed in a change orientation wherein the surgical handpiece 300 is oriented vertically or substantially vertically with the tool 500 at the distal portion of the surgical handpiece 300 pointed upwardly; and, as shown in FIG. 3C, when one or more surgical handpieces 300 are on standby or otherwise not in use, the one or more surgical handpieces 300 may be oriented in a standby orientation wherein the one or more surgical handpieces 300 are substantially horizontal (and disposed on a surgical tray 600).

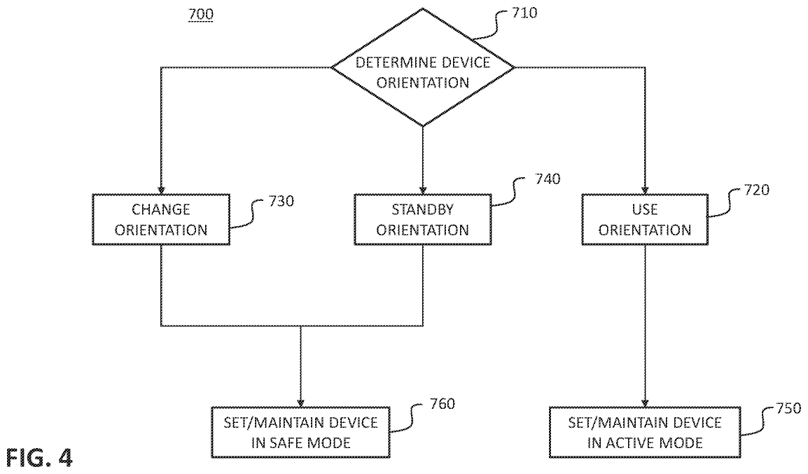

Continuing with reference to FIGS. 3A-3C, and with additional reference to FIGS. 1 and 4, as noted above, the orientation sensor 330 of each surgical handpiece 300 is connected to console 100 to enable determination of the orientation of the surgical handpiece 300. The determined orientation of each surgical handpiece 300 may be utilized to set a mode of the surgical handpiece 300 such as, for example, between an active mode and a safe mode. In the active mode, for example, the surgical handpiece 300 can be activated, controlled, and/or otherwise utilized without any added limitation(s). In the safe mode, on the other hand, activation, control, and/or other use of the surgical handpiece 300 may be inhibited, limited, or require additional action (such as an additional activation or other confirmatory action, clearance/acknowledgement of a warning, etc.). As illustrated with respect to process 700 of FIG. 4, console 100 or other suitable component(s) may first determine, as indicated at 710, the orientation of the one or more surgical handpieces 300. The determination at 710 may indicate, for example, that the surgical handpiece 300 is in the use orientation (FIG. 3A) as indicated at 720, that the surgical handpiece 300 is in the change orientation (FIG. 3B) as indicated at 730, or that the surgical handpiece 300 is in the standby orientation (FIG. 3C) as indicated at 740.

Depending upon the determined orientation, console 100 may maintain or set the mode of the surgical handpiece 300, e.g., between the active mode and the safe mode. For example, as indicated at 750, if the surgical handpiece 300 is determined to be in the use orientation (FIG. 3A), console 100 may maintain or set the surgical handpiece 300 to the active mode. On the other hand, as indicated at 760, if the surgical handpiece 300 is determined to be in the change orientation (FIG. 3B) or standby orientation (FIG. 3C), console 100 may maintain or set the surgical handpiece 300 to the safe mode. The determination at 710 of the orientation of the surgical handpiece 300 may be made continuously, periodically at prescribed intervals, randomly, and/or upon occurrence of an event, e.g., motion detection, connection/disconnection of a surgical handpiece 300, activation/deactivation of a surgical handpiece 300, etc. In aspects, determining that the surgical handpiece 300 is in the standby orientation (FIG. 3C) may also require detecting no motion of the surgical handpiece 300, e.g., using the sensed orientation data; determining that the surgical handpiece 300 is in the change orientation (FIG. 3B) may also require detecting no or minimal motion (below a threshold) of the surgical handpiece 300, e.g., using the sensed orientation data; and/or determining that the surgical handpiece 300 is in the use orientation (FIG. 3A) may also require detecting sufficient motion (above a threshold) of the surgical handpiece 300, e.g., using the sensed orientation data.

Referring to FIG. 5, in conjunction with FIGS. 1 and 3A-3C, as noted above, console 100 may include multiple surgical handpieces 300 each connected to one of the ports 140 thereof. An accessory device such as, for example, footswitch 200, may also be connected to one of the ports 140 of console 100. Console 100 and/or footswitch 200 may be utilized to control the various surgical handpieces 300. However, console 100 and footswitch 200 may only be configured to control one or a limited number of the surgical handpieces 300 at any given time. For example, one of the processors 160 of console 100 may selectively assign footswitch 200 to a particular surgical handpiece 300 depending upon user input and/or other feedback received. Similarly, console 100 may display information relating to one or more surgical handpieces 300 among the group of connected surgical handpieces 300, enable adjustment of settings of one or more surgical handpieces 300 among the group of connected surgical handpieces 300, and/or control activation of one or more surgical handpieces 300 among the group of connected surgical handpieces 300 depending upon user input and/or other feedback received.

With additional reference FIG. 6, the surgical handpiece(s) 300 to be controlled by console 100 and/or footswitch 200 may be determined according to process 800. Initially, as indicated at 810, the active surgical handpiece(s) 300 is determined. This may be accomplished by determining which, if any, surgical handpieces 300 are disposed in the active mode, as detailed above. Alternatively, sensed orientation data provided by the orientation sensors 330 of the surgical handpieces 300 may be utilized by the processor 160 of console 100, e.g., by comparison with previous sensed orientation data, to determine which surgical handpieces 300 are active based on whether a surgical handpiece 300 is stationary or whether a surgical handpiece 300 is moving (or was recently moved). Recent movement may be determined where motion is detected within an immediately preceding period of time (e.g., 3 second, 5 seconds, or 10 seconds).

Once the active surgical handpieces 300 have been determined, console 100 may output a suitable signal to those active surgical handpieces 300 to provide an output from output devices 340 thereof, e.g., a particular color LED, an audible tone, etc., as indicated at 820. The active surgical handpiece determination in 810 may be performed continuously, periodically at prescribed intervals, randomly, and/or upon occurrence of an event, e.g., motion detection. Upon motion detection, for example, an output may be provided from console 100 to the output device 340 of a surgical handpiece 300 when motion of that surgical handpiece 300 is detected, thus indicating to the user whether that surgical handpiece 300 is controllable by console 100 and/or footswitch 200. Alternatively or additionally, outputs may be provided to the surgical handpieces 300 that are not active or in motion. In aspects, an LED of the output device 340 may be illuminated green when the surgical handpiece 300 is controllable (or active) while an LED of the output device 340 may be illuminated red when the surgical handpiece 300 is not controllable (or not active). Illumination of an LED (or other suitable output) indicating which surgical handpieces 300 are controllable or active (in active mode) versus which surgical handpieces 300 are not active (in safe mode) or not controllable may be temporary, periodic, or continuous.

Continuing to 830, once the active surgical handpieces 300 have been determined (and, in aspects, suitable outputs indicating the same have been provided), console 100 assigns controls and/or settings thereof, and/or controls and/or settings of footswitch 200 to the one or more active surgical handpieces 300. As indicated in 840, inactive surgical handpieces 300 are set to safe mode, similarly as detailed above. Thus, as detailed above, console 100 and/or footswitch 200 can be automatically assigned to an appropriate surgical handpiece(s) 300 and/or the status of each surgical handpiece 300, the active surgical handpiece(s) 300, and/or the inactive surgical handpiece(s) can be readily determined.

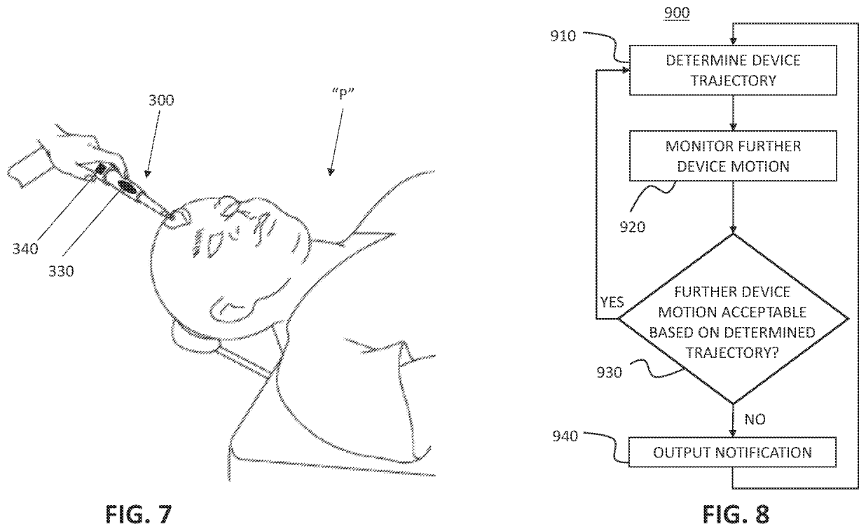

Turning to FIG. 7, in conjunction with FIG. 1, as noted above, the orientation data provided from the orientation sensor 330 of a surgical handpiece 300 to console 100 may be utilized by a processor 160 (FIG. 5) of console 100 to determine a direction of motion of the surgical handpiece 300, e.g., based upon tracking the orientation data as a function of time. Tracking the orientation data as a function of time, e.g., as surgical handpiece 300 is utilized to perform a surgical task on tissue of a patient “P,” may also enable determination of a trajectory of the surgical handpiece 300. It is noted that the trajectory of the surgical handpiece 300 is not limited to a straight line or curve, but can include a repeating pattern such as, for example, a helical motion, a circular motion, a back and forth motion, a side to side motion, etc.

With additional reference to FIG. 8, in use, process 900 may be utilized to enable monitoring of the trajectory of the surgical handpiece 300. Initially, e.g., during an initial period of use, the trajectory of the surgical handpiece 300 is determined, as indicated at 910 and as detailed above. Once the trajectory of the surgical handpiece 300 is determined, further motion thereof is monitored (again based on the orientation data provided from the orientation sensor 330 of a surgical handpiece 300 to console 100) and the processor 160 (FIG. 5) of console 100 compares the further motion data to the determined trajectory, as indicated at 920. If the further motion is aligned with the determined trajectory, sufficiently close to the determined trajectory (such as, for example, within a cone of deviation or other acceptable limits), or otherwise acceptable based on the determined trajectory, “YES” at 930, the process reverts to 910 without further action. On the other hand, if the further motion sufficiently deviates from the determined trajectory or otherwise exceeds acceptable limits, “NO” at 930, the process proceeds to 940, wherein an output notification is provided such as, for example, an output from output device 340 of the surgical handpiece 300 in the form of an illuminated LED (e.g., solid red, blinking red, etc.) and/or an audible tone or series of tones. In aspects, the output provided is scaled or otherwise modified based on the severity of deviation from the determined trajectory, e.g., a yellow light for minor deviation and a red light for a major deviation; and/or a tone at a relatively low volume for a minor deviation and a tone at a relatively high volume for a major deviation. In this manner, a user is alerted that the surgical handpiece 300 no longer follows the determined trajectory and, in aspects, the extent to which the surgical handpiece 300 is off-trajectory.

While several aspects of the present disclosure have been shown in the drawings, it is not intended that the present disclosure be limited thereto, as it is intended that the present disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto.

What is claimed is:

1. A surgical system, comprising:
a surgical handpiece, including:
a handle housing;
a connector extending from the handle housing, the connector adapted to connect the surgical handpiece to a console;
a tool releasably coupled with the handle housing; and
an orientation sensor disposed within the handle housing, the orientation sensor configured to facilitate determination of an orientation of the surgical handpiece from between at least a use orientation and a change orientation.

2. The surgical system according to claim 1, wherein the orientation sensor includes at least one of an accelerometer, a gyroscope, or a magnetometer.

3. The surgical system according to claim 1, wherein the orientation sensor is further configured to facilitate determination of the orientation of the surgical handpiece from between at least the use orientation, the change orientation, and a standby orientation.

4. The surgical system according to claim 1, wherein the surgical handpiece further includes an output device configured to provide at least one of an audible output, a visual output, or a haptic output.

5. The surgical system according to claim 1, further comprising the console, wherein the orientation sensor is configured to transmit orientation sensor data to the console and wherein a processor of the console is configured to determine the orientation of the surgical handpiece based at least in part on the orientation sensor data.

6. The surgical system according to claim 5, where the processor of the console is further configured to: set or maintain the surgical handpiece to an active mode when the determined orientation is the use orientation, and set or maintain the surgical handpiece to a safe mode when the determined orientation is the change orientation.

7. The surgical system according to claim 6, wherein at least some functionality of the surgical handpiece is inhibited in the safe mode.

8. A surgical system, comprising:
a console; and
a plurality of surgical handpieces connected to the console, each surgical handpiece including:
a handle housing;
a connector extending from the handle housing, the connector connecting the surgical handpiece to the console;
a tool releasably coupled with the handle housing; and
an orientation sensor disposed within the handle housing,
wherein the console includes a processor configured to receive orientation sensor data from the orientation sensor of each surgical handpiece of the plurality of surgical handpieces and to determine at least one active surgical handpiece of the plurality of surgical handpieces and at least one inactive surgical handpiece of the plurality of surgical handpieces based on the orientation sensor data.

9. The surgical system according to claim 8, wherein the console enables remote control of the at least one active surgical handpiece and inhibits remote control of the at least one inactive surgical handpiece.

10. The surgical system according to claim 9, wherein the remote control includes at least one of control at the console or control at an accessory attached to the console.

11. The surgical system according to claim 10, wherein the accessory is a footswitch.

12. The surgical system according to claim 8, wherein the processor of the console is configured to determine whether each surgical handpiece of the plurality of surgical handpieces is in motion or has recently been in motion based on the orientation sensor data to determine the at least one active surgical handpiece and the at least one inactive surgical handpiece.

13. The surgical system according to claim 8, wherein each surgical handpiece of the plurality of surgical handpieces further includes an output device configured to provide at least one of an audible output, a visual output, or a haptic output.

14. The surgical system according to claim 13, wherein the processor of the console is configured to direct the output device of the at least one active surgical handpiece to output an indication that the at least one active surgical handpiece is active.

15. The surgical system according to claim 8, wherein the processor of the console is configured set or maintain the at least one inactive surgical handpiece to a safe mode.

16. A surgical system, comprising:
a console; and
a surgical handpiece connected to the console, the surgical handpiece including:
a handle housing;
a connector extending from the handle housing, the connector connecting the surgical handpiece to the console;
a tool releasably coupled with the handle housing; and
an orientation sensor disposed within the handle housing,
wherein the console includes a processor configured to receive orientation sensor data from the orientation sensor of the surgical handpiece and to determine a trajectory of motion of the surgical handpiece based thereon.

17. The surgical system according to claim 16, wherein the orientation sensor includes at least one of an accelerometer, a gyroscope, or a magnetometer.

18. The surgical system according to claim 16, wherein the processor of the console is further configured to determine, based on further orientation sensor data received from the orientation sensor of the surgical handpiece, whether the surgical handpiece deviates from the determined trajectory.

19. The surgical system according to claim 18, wherein the surgical handpiece includes an output device, and wherein the processor of the console is configured to instruct the output device to provide an output indicating that the surgical handpiece has deviated from the determined trajectory when it is determined that the surgical handpiece has deviated from the determined trajectory.

20. The surgical system according to claim 19, wherein the output device is configured to provide at least one of an audible output, a visual output, or a haptic output.
